(19) United States
(12) Patent Application Publication
(10) Pub. No.: US 2022/0031293 A1
(43) Pub. Date: Feb. 3, 2022

(54) USER INTERFACE FOR BIOPSY DEVICE

(71) Applicant: Devicor Medical Products, Inc., Cincinnati, OH (US)

(72) Inventors: Andrew T. Robinson, Cincinnati, OH (US); Emmanuel V. Tanghal, Mason, OH (US); Clayton C. Barclay, III, Milford, OH (US)

(21) Appl. No.: 17/504,854

(22) Filed: Oct. 19, 2021

Related U.S. Application Data
(63) Continuation of application No. PCT/US2020/029677, filed on Apr. 24, 2020.
(60) Provisional application No. 62/839,132, filed on Apr. 26, 2019.

(51) Int. Cl. A61B 10/02; A61B 8/00

(52) U.S. Cl. CPC A61B 10/0283; A61B 8/4405; A61B 8/469; A61B 8/463; A61B 8/54

(57) ABSTRACT

A unitary control module provides features of an ultrasound system and a biopsy system. A lower module positioned near the bottom of the cart may include power supplies, vacuum components, and biopsy controller components, while an upper module positioned near the top of the cart may include a display, user input devices, and an ultrasound controller. The components of the upper module and lower module may be connected via cabling, hoses, or other connections that run within or along a height adjustable channel member that supports the upper module. The ultrasound controller may be configured to serve as a primary processor for rendering a user interface via the display, and handling user inputs. The ultrasound controller is in communication with the biopsy controller, and handles interpreting and converting inputs and outputs from the biopsy controller in order to provide biopsy status and control features via the user interface.

### PRIORITY

This application claims priority to U.S. Provisional Application Ser. No. 62/839,132, entitled “User Interface for Biopsy Device,” filed on Apr. 26, 2019, the disclosure of which is incorporated by reference herein.

### BACKGROUND

A biopsy is the removal of a tissue sample to examine tissue for signs of cancer or other disorders. Tissue samples are obtained in a variety of ways using various medical procedures involving a variety of the sample collection devices. For example, biopsies may be open (surgically removing tissue) or percutaneous (e.g. by fine needle aspiration, core needle biopsy or vacuum assisted biopsy). After the tissue sample is collected, the tissue sample is analyzed at a lab (e.g. a pathology lab, biomedical lab, etc.) that is set up to perform the appropriate tests (such as histological analysis).

A biopsy suite includes a number of devices usable to aid in the collection of tissue samples, which may include patient support structures, x-ray imaging devices, ultrasound imaging devices, biopsy devices, and various control systems. An x-ray imaging device may be used to capture pre-procedure imaging of a patient, which may aid in identifying tissue from which samples should be collected, while ultrasound imaging may be used during a tissue collection procedure to aid in the positioning and use of a percutaneous biopsy device. Such a procedure may be performed by a physician with a biopsy device held or guided in one hand, and an ultrasound transducer simultaneously held or guided by the other hand. Since the ultrasound transducer and the biopsy device have separate and independent control systems, such simultaneous uses may be disadvantageous for a physician where, for example, they are focusing on an ultrasound display during the procedure, and may need to check the status of, or change the configuration of the biopsy device. When shifting focus between a display and user interface for an ultrasound transducer, and a display and user interface for a biopsy device, the physician may become confused or may be unable to quickly identify the proper user interface to interact with in order to perform a specific task. This confusion or delay could lead to an increased risk of error, or could decrease the patient's comfort or satisfaction with a procedure. These separate control systems may also be disadvantageous in that they are often mounted on separate carts or workspaces, which then must be positioned closely together to allow for simultaneous use. This can lead to a procedure environment that is cluttered with equipment and cabling, which can reduce the mobility and effectiveness of clinicians working therein.

While several systems and methods have been made and used for obtaining and processing a biopsy sample, it is believed that no one prior to the inventor has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements. In the drawings some components or portions of components are shown in phantom as depicted by broken lines.

FIG. 1 depicts an exemplary biopsy suite;

FIG. 2 depicts a schematic diagram of an exemplary biopsy controller usable with the biopsy suite of FIG. 1;

FIG. 3 depicts a schematic diagram of an exemplary ultrasound controller usable with the biopsy suite of FIG. 1;

FIG. 4 depicts a perspective view of an exemplary unitary control module usable with the biopsy suite of FIG. 1;

FIG. 5 depicts a schematic diagram of an exemplary unitary control module;

FIG. 6 depicts a flowchart of an exemplary set of steps that may be performed with the unitary control module of FIG. 5 to provide a combined user interface;

FIG. 7 depicts a schematic diagram of an alternate exemplary unitary control module;

FIG. 8 depicts a flowchart of an exemplary set of steps that may be performed with the unitary control module of FIG. 7 to provide a combined user interface; and

FIG. 9 depicts a screenshot of an exemplary user interface that may be provided by the unitary control module of FIGS. 4, 5, or 7.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

I. Exemplary Biopsy Suite

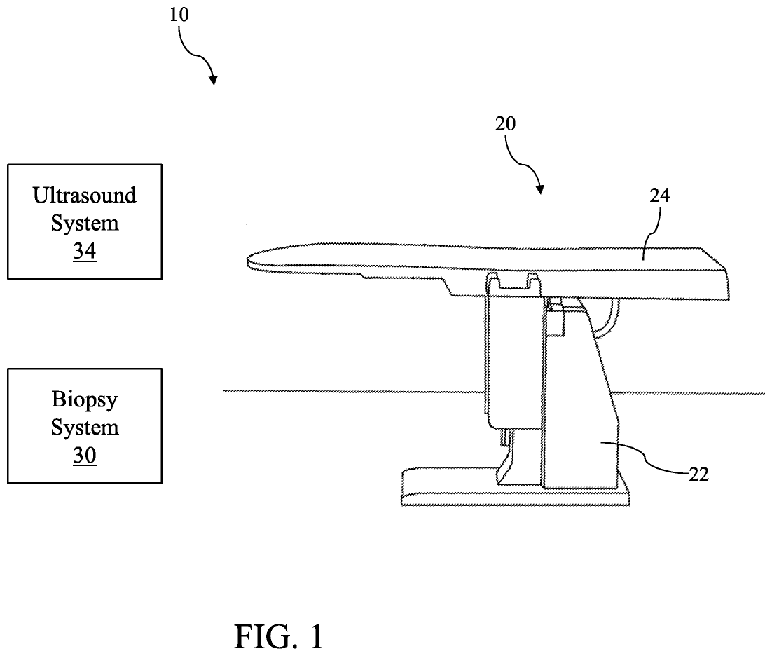

FIG. 1 shows an exemplary biopsy suite (10). Suite (10) comprises a support assembly (20). Generally, support assembly (20) is operable to support a patient during a procedure. Support assembly (20) of the present example includes a base assembly (22) supporting a patient table (24). Generally, base assembly (22) is adjustable to position table (24) to allow a physician to interact with patient during a procedure. A biopsy system (30) is operable by a physician to extract tissue using a biopsy device. A user may interact with the biopsy system (30) to activate and configure the operation of the biopsy device, which may include adjusting the size of a cutting aperture, rotating a tissue sample holder, adjusting a vacuum power, and other changes. Merely exemplary biopsy system components, including exemplary biopsy devices and exemplary tissue sample holders, are disclosed in U.S. Pat. Pub. No. 2014/0039343, entitled “Biopsy System,” filed Aug. 12, 2013, the disclosure of which is incorporated by reference herein. Extraction of tissue samples with the biopsy system (30) may be aided by the use of imaging techniques, which may include an ultrasound system (34), to provide intra-procedure imaging of the target site.

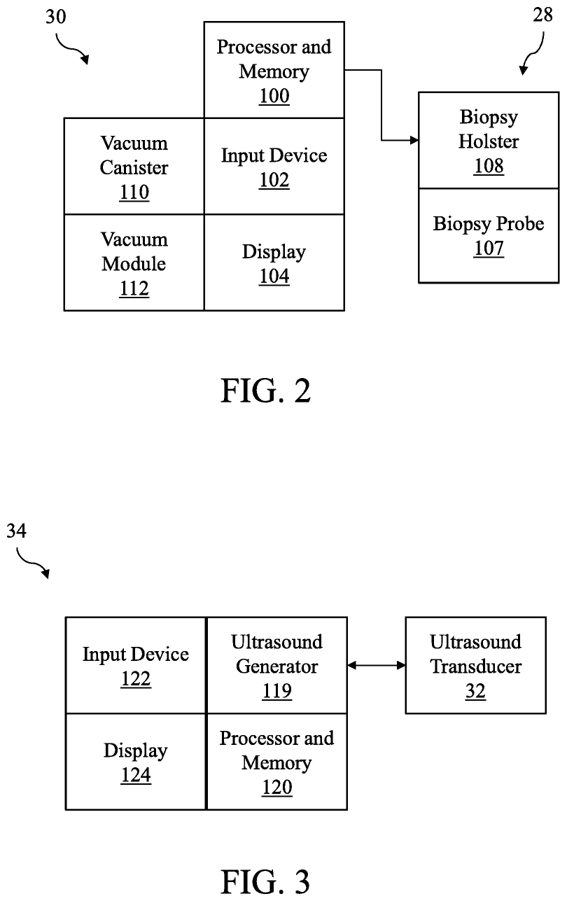

FIG. 2 shows a schematic diagram of an exemplary biopsy system, such as the biopsy system (30). The biopsy system (30) includes a biopsy holster (108) which couples with a biopsy probe (107) to form a biopsy device (28). The biopsy probe (107) may itself be designed to be disposable and may lack electronic components, while the biopsy holster (108) is reusable and provides power to drive mechanical portions of the biopsy device (107). A display (104) may be an LCD, LED, or other display type, and may be configured to provide status information relating to the biopsy device (28), such as aperture size, vacuum power, tissue sample holder position, and other related information. An input device (102) may be one or more of a touchscreen, keypad, keyboard, mouse, foot pedal, or other devices that may receive input from a user to control the biopsy device (28), which may include varying aperture size, vacuum power, tissue sample holder position, or other configurations.

A vacuum module (112) provides suction to the biopsy device (28) to collect tissue, saline, and bodily fluids, which may be transferred to a vacuum canister (110), which is adapted to be safely removed and exchanged with an empty canister during or between procedures. The vacuum module (112) may also provide suction to collect tissue samples and provide them to a collection mechanism such as a tissue sample holder. A processor and memory (100) are operable to store and execute instructions to operate other components of the biopsy system (30), including rendering an interface via the display (104), receiving and acting upon user input from the input device (102), and controlling the operation of the biopsy device (28). The biopsy system (30) may include other features, such as network interfaces, data connections (e.g., USB, serial), power connections, batteries, and other similar devices as will be apparent to one of ordinary skill in the art in light of this disclosure.

FIG. 3 depicts a schematic diagram of the ultrasound system (34). The ultrasound system (34) includes an ultrasound generator (119) capable of producing control signals to cause a transducer in the ultrasound transducer (32) to generate and emit ultrasound frequencies appropriate for ultrasound imaging. A display (124) may be an LCD or other display type, and may be configured to display status information relating to the ultrasound system (34), as well as the results of ultrasound imaging of a targeted site. An input device (122) may be a touchscreen, keypad, keyboard, mouse, or other user input, and may be interacted with by a user to configure and control the ultrasound system (34). A processor and memory (120) are operable to store and execute instructions to operate other components of the ultrasound system (34), which may include rendering an interface via the display (124), transmitting images or data to a printing device, receiving and acting upon inputs from the input device (122), controlling the ultrasound generator (119), and receiving an interpreting incoming ultrasound signals via the ultrasound transducer (32) to produce ultrasound images.

II. Exemplary Unitary Control Module

The biopsy system (30) and the ultrasound system (34) may be disadvantageous for simultaneous use because each includes a separate display and input device. For example, during use a physician may be viewing ultrasound images on the display (124), and be unaware of an error message or other important biopsy device status information being displayed on the display (104). As another example, a physician may be viewing ultrasound images on the display (124), and may wish to change the vacuum power of the biopsy device (28). This may lead to confusion where the physician initially attempts to change the vacuum power using the input device (122), before realizing that they must instead view the display (104) and interact with the input device (102).

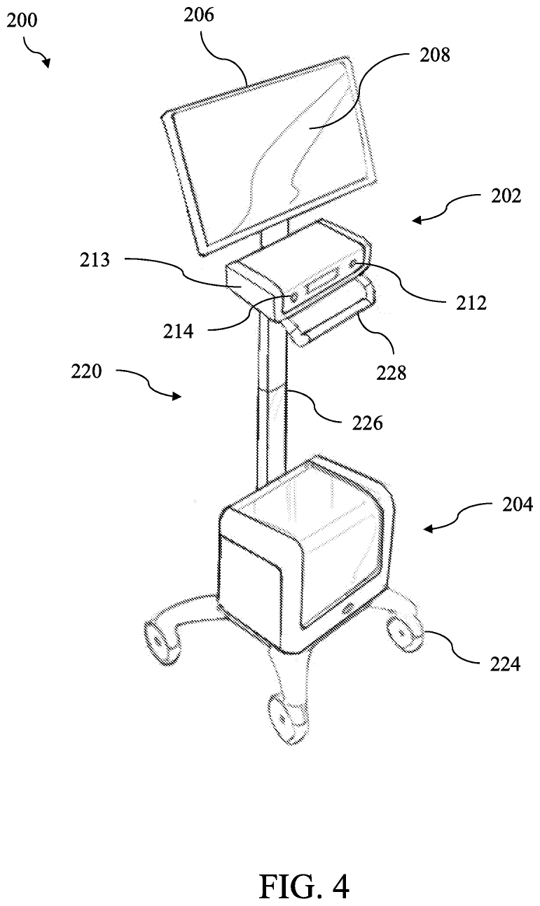

FIG. 4 depicts a perspective view of a unitary control module (200) that combines many or all of the features of the biopsy system (30) and the ultrasound system (34) into a single system, which provides a number of advantages. The unitary control module (200) includes a cart (220) with a lower module (204) and an upper module (202). The upper module (202) includes or is proximate to a display (206), which may be an LCD, LED, or other display type capable of displaying information similar to the display (104) and the display (124). The display (206) includes or is proximate to an input device (208) such as a touchscreen that is capable of receiving user inputs, but may also include a keyboard, mouse, or other input device attached to or positioned near the display (206).

The display (206) and input device may connect to an upper module hub (213), which includes a biopsy connection (212), an ultrasound connection (214), and other internal components described in more detail below. The biopsy connection (212) may include a biopsy holster, such as the biopsy holster (108), which may receive and operate a device such as the biopsy probe (107), and which may include connections for receiving suctioned tissue and bodily fluids from the biopsy probe (107), as has been described. The ultrasound connection (214) may connect to the ultrasound transducer (32) to provide power and control, as has been described.

The lower module (204) includes internal components usable to operate the unitary control module (200), which may include power supplies, vacuum modules, and other components that will be described in more detail below. The upper module (202) and the lower module (204) are mounted the cart (220), and connections between the upper module (202) and the lower module (204), which may include electrical connections, data connections, vacuum hosing, and other connections, may be routed through an adjustable channel member (226). The adjustable channel member (226) supports the upper module (202), and provides an internal channel through which connections and cabling may be routed. The adjustable channel member (226) may be height adjustable so that the upper module (202) can be positioned comfortably for a particular physician or other user to interact with. The cart (220) also includes a set of wheels (224) and a handle (228) to aid in transportation of the unitary control module (200).

As shown in FIG. 4, the unitary control module (200) includes a single display, the display (206), which may be configured to provide a combined user interface, which may include a graphical user interface configured to display information relating to the biopsy system and the ultrasound system, and receive user inputs (e.g., via a touchscreen, mouse, keyboard, or other input device) for interacting with the biopsy system and the ultrasound system. While some implementations of the unitary control module (200) may include more than one display, an implementation having only a single display or single screen may be advantageous to reduce weight and overall dimensions, and prevent confusion as to which screen or display a user should be viewing or interacting with during use.

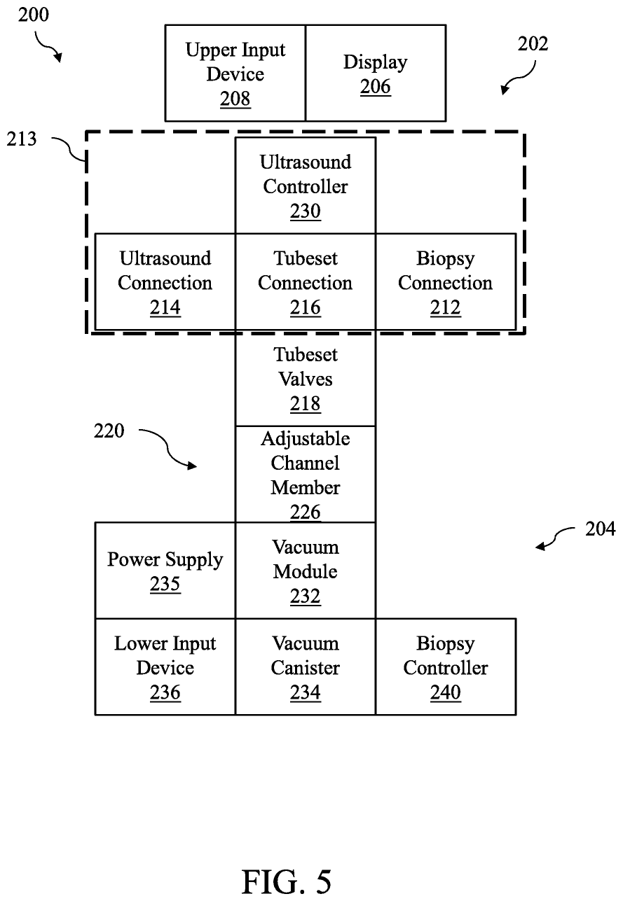

FIG. 5 depicts a schematic diagram of the unitary control module (200). The upper module (202) includes an ultrasound controller (230), which includes one or more components of an ultrasound system such as the ultrasound system (34). The ultrasound controller (202) may be within the upper module hub (213), and may include a processor and memory (e.g., such the processor and memory (120)) and an ultrasound generator (e.g., such as the ultrasound generator (119)). An ultrasound transducer (32) connected to the ultrasound connection (214) will be paired with the ultrasound controller (230), and the ultrasound controller (230) may also be coupled with and configured to operate with display (206) and the upper input device (208), which may include providing a graphical user interface and receiving and acting upon user inputs. The upper module hub (213) may also include a tubeset connection (216), which may be part of or separate from the biopsy connection (212), and which is configured to receive tissue and bodily fluid suction from the biopsy device (28) and route it, depending upon the configuration of a set of tubeset valves (218), to a vacuum module (232) or elsewhere. The tubeset valves (218) may be manually adjustable in some implementations, or may be automatically adjustable by the ultrasound controller (230) or biopsy controller (240), or both.

In the implementation of FIG. 5, the adjustable channel member (226) may contain a connection from a power supply (235) in the lower module (204) that provides power to the ultrasound controller (230), display (206), and other devices of the upper module (202). A set of vacuum hoses that couple the tubeset connection (216) with the vacuum module (232) may also be contained within the adjustable channel member (226), or may be attached to and run on the outside of the member (226), either on the surface or partially or fully obscured by a shielding, casing, or other routing means.

The vacuum module (232) is operable to provide suction and transfer tissue and bodily fluids from the biopsy device (28) to the vacuum canister (234), which is adapted to store excess saline and bodily fluids, and allow for easy removal and disposal. The lower module (204) also includes a biopsy controller (240) that is operable to control the vacuum module (232), and to control the biopsy device (28) that are coupled with the biopsy connection (212). The lower module (204) may also include a lower input device (236), which may include a foot pedal or other input device, which may be interacted with by a user to provide input to the biopsy controller (240), the ultrasound controller (230), or both.

As can be seen in FIG. 5, the unitary control module (200) includes both the ultrasound controller (230) as well as the biopsy controller (240), but does not include separate displays and user input devices for each of those systems. In such an implementation, the ultrasound controller (230) may be configured to provide primary processing for both providing the ultrasound capabilities, as well as for providing the user interface via the display (206) and handling user inputs (e.g., via the upper input device (208), the lower input device (236), or both). The ultrasound controller (230) is also coupled with the biopsy controller (240), such that status information, user interface features, control features, and user inputs may be exchanged between the controllers. This may include the ultrasound controller (230) receiving status information describing the biopsy system (e.g., the biopsy device, holster, probe, and controller), and displaying that information via the display (206) as part of the user interface for the ultrasound system. This may also include the upper input device (208) being used to provide a user input, such as a configuration change intended for the biopsy system (e.g., a change in vacuum power), that is received by the ultrasound controller (230) via a user interface provided by the ultrasound controller (230). In this manner, a single user interface may be provided that includes status information and controls for both ultrasound features as well as biopsy features, rather than splitting such functionality across two displays (e.g., the display (124) and the display (104)) or two input devices (e.g., the input device (122) and the input device (102)).

Since the ultrasound controller (230) handles rendering of a combined user interface for both the ultrasound features as well as the biopsy features, the ultrasound controller (230) may be configured to receive, interpret, and convert outputs from the biopsy controller (240), and inputs to the biopsy controller (240), such that the systems can interface directly with each other. As an example, a user input may be received via the upper input device (208) as a string value, and may be converted to an integer value, binary value, or other encoded value that is usable by the biopsy controller (240) before being transmitted to the biopsy controller (240). Similarly, output from the biopsy controller (240) may be provided as binary values or other encoded values, and may be converted by the ultrasound controller (230) to string values, graphical representations, or other objects before being displayed via the display (206).

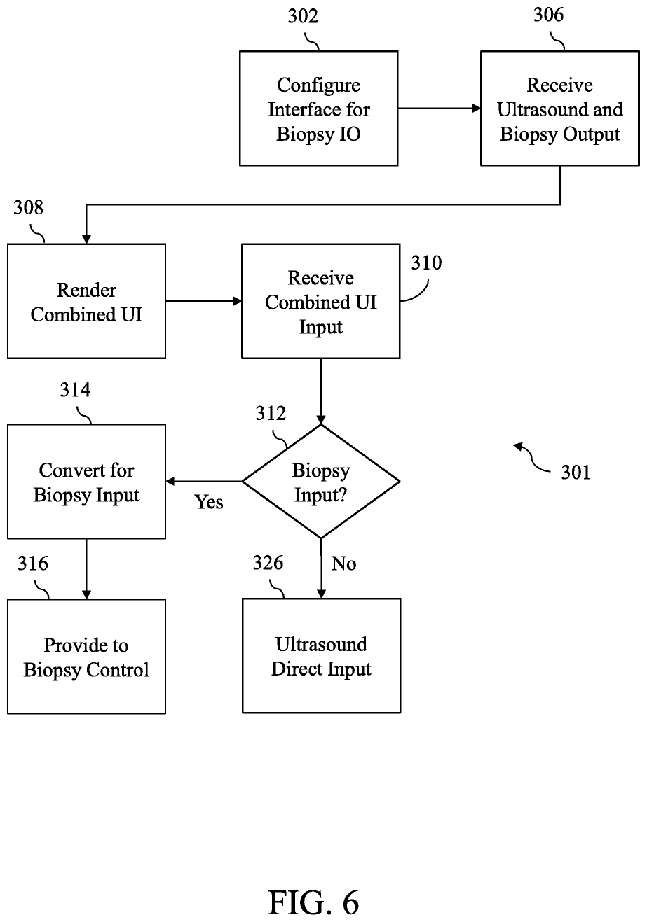

As an example, FIG. 6 shows a flowchart of an exemplary set of steps (301) that may be performed by the ultrasound controller (230) to provide a combined user interface. This may include configuring (block 302) the user interface for input/output with the biopsy system, which may include placing the ultrasound controller (230) and the biopsy controller (240) in communication with each other, and configuring the ultrasound controller (230) to receive, interpret, and convert input and output for the biopsy controller (240). As an example, this may include creating or modifying software and configurations for the ultrasound controller (230) to handle input and output with the biopsy controller (240), and provide a user interface for both controllers.

Once configured (block 302), output may be received from the ultrasound controller (230), the biopsy controller (240), or both, which may include status information for each controller and any attached probes, holsters, or devices. A combined user interface may then be rendered (block 308) via the display (206) based upon output related to the ultrasound and biopsy features. When an input is received (block 310) via the combined user interface, it may be determined whether it is a biopsy input (block 312) that is intended for the biopsy controller (240). This determination may be made based upon the manner in which the input is received (e.g., a selection via the combined user interface of an ultrasound control can be differentiated from a selection of a biopsy control), or may be made based upon an analysis of the input value itself (e.g., by examining the string, integer or other value and comparing to a set of known input formats or types).

Where the received (block 310) input is a biopsy input (block 312), the input can be converted (block 314) into a format that is usable by the biopsy controller (240), and provided (block 316) to the biopsy controller (240). As an example, an input received (block 310) via the combined user interface may be a value associated with a key that identifies the user interface element it was received from (e.g., a value “1” indicating a feature has been turned on, and a value “biopsyVacuum” indicating that the value was received as a result of a user selecting an interface feature associated with enabling or disabling the suction feature of the biopsy device). Such an input may be converted (block 314) into a binary value that is usable by the biopsy controller (240) to identify the vacuum feature and change in configuration, and provided to the biopsy controller (block 316) to cause the desired change (e.g., such as enabling the vacuum module (232)). Where it is determined (block 312) that the input is not a biopsy input, it can be determined (block 326) to be a direct input to the ultrasound system, such as enabling the ultrasound transducer (32) or modifying the ultrasound transducer depth. A direct input may not require conversion in the same manner as inputs intended for the biopsy controller (240), since there may be no need to interface between two distinct control systems.

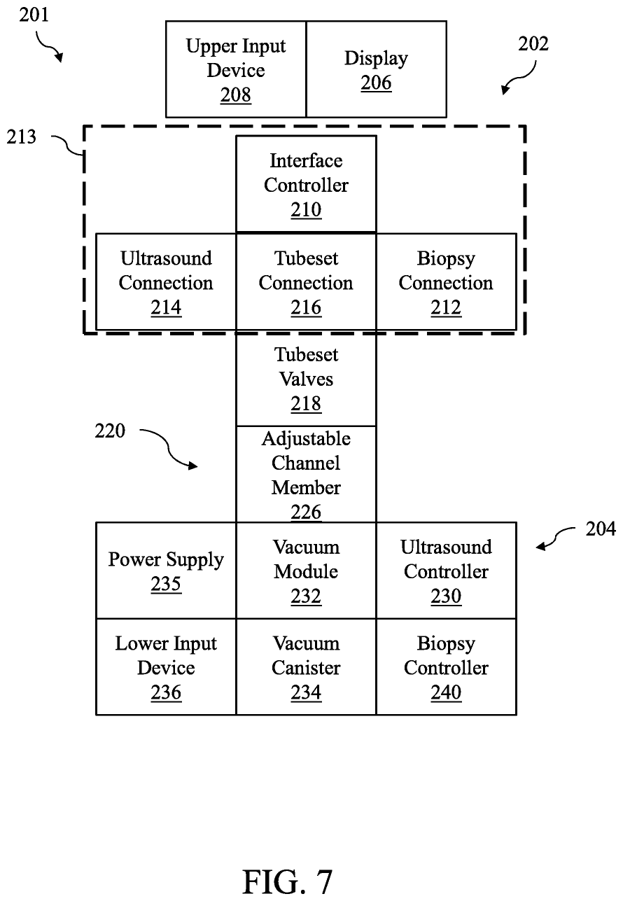

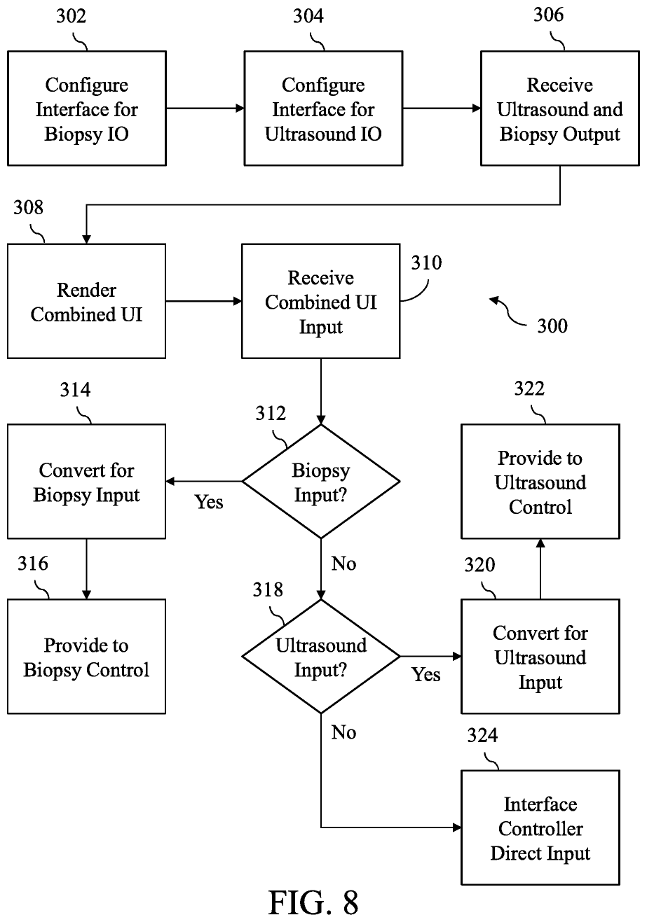

While FIGS. 5 and 6 show the unitary control module (200) implemented with the ultrasound controller (230) providing primary processing and interfacing for the entire system, other implementations exist and will be apparent to one of ordinary skill in the art. As an example, FIGS. 7 and 8 show an alternative unitary control module (201) that includes the ultrasound controller (230) and the biopsy controller (240) in the lower module (204), with an interface controller (210) positioned in the upper module (202) that is dedicated to rendering and providing a user interface via the display (206) based upon receiving, interpreting, and converting inputs and outputs from the ultrasound controller (230) and the biopsy controller (240). The interface controller (210) may include a processor, memory, a network interface, and various data connections, and may be, for example, a rack mount computer, a single board computer, or another computing device. The interface controller (210) could be configured to receive outputs from and provide inputs to the ultrasound controller (230), the biopsy controller (240), and other devices, and interpret and convert those inputs and outputs to render a combined user interface and route user inputs to the appropriate device.

By implementing the interface controller (210) as described, a variety of different ultrasound controllers and biopsy controllers, from varying manufacturers or other sources, may be added to the unitary control module (201) and configured to operate with the interface controller (210). This may be useful where, for example, a manufacturer provides the unitary control module (201) with only components of the upper module (202) chosen and pre-configured, and an end-user of the system may provide or select their own biopsy system (e.g., the biopsy controller (240), the vacuum module (230), etc.) and ultrasound system (e.g., the ultrasound controller (230), the ultrasound transducer (32)) to be added to the system. As another example, a manufacturer may provide the unitary control module (201) with the biopsy system pre-configured, and an end-user of the system may provide or select their own ultrasound system. In such an example, a variety of off-the-shelf solutions for transducer capabilities may be integrated with proper configuration of the interface controller (210). One such example may include the application-based ultrasound devices such as those provided by UAB “TELEMED” of Vilnius, Lithuania, as part of the “ArtUs” platform, under model numbers C5-2H60-A5, C7-3N50-A5, and others.

The interface controller (210) may be pre-configured to support inputs and outputs from a variety of systems and connected devices that a user may select from, or such configurations for interpreting, converting, and routing input and output may be configured on an as needed-basis, or through an interface controller training process that may include, for example, connecting a chosen ultrasound system, and then performing various functions with the ultrasound system while specifying each function to the interface controller (210).

FIG. 8 shows an exemplary set of steps (300) that may be performed by the interface controller (210) to provide a combined user interface. The interface controller (210) could be configured (block 302) to handle inputs and outputs for the biopsy controller (240), and could also be configured (block 304) to handle inputs and outputs for the ultrasound controller (230). The interface controller (210) would receive (block 306) output from the biopsy system and the ultrasound system, and interpret and convert that output as configured to render (block 308) the combined user interface via the display (206). When receiving (block 310) input via the combined user interface, similar to the descriptions in the context of FIG. 5, it would be determined if a particular input was a biopsy input (block 312), an ultrasound input (block 318), or a direct interface controller input (block 324). Inputs intended for the biopsy system would be converted (block 314) to be usable by that system, and provided (block 316) to the biopsy controller (240). Inputs intended for the ultrasound system would be converted (block 320) to be usable by that system, and provided (block 322) to the ultrasound controller (230). Inputs intended for the interface controller (block 324) might include configuration changes for the interface controller (210) itself, which could include configuration changes specifying the type of ultrasound or biopsy system installed in the unitary control module (201), or other changes specific to the function provided by the interface controller (210).

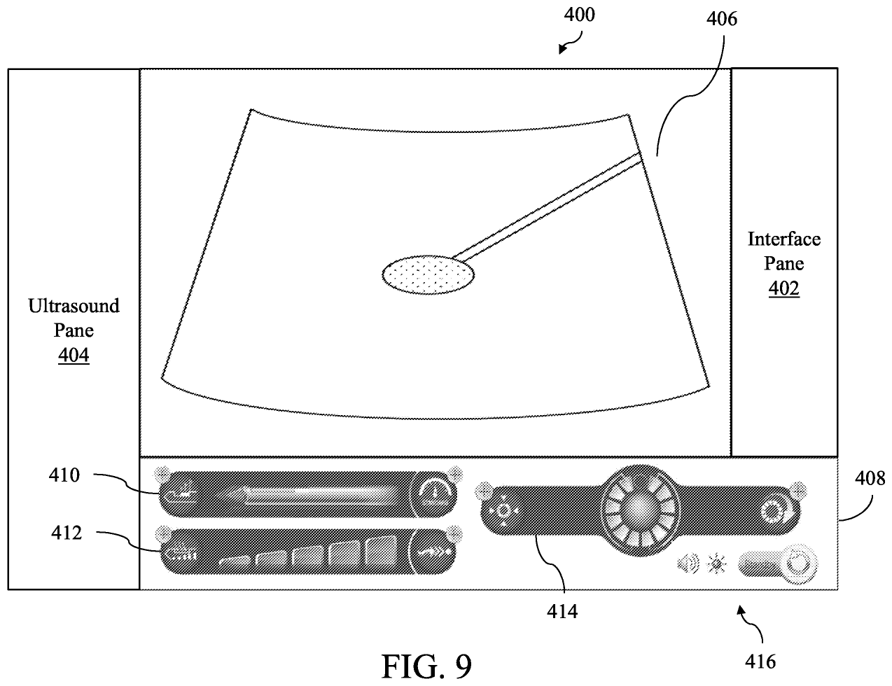

FIG. 9 depicts an exemplary combined user interface (400) usable to view information and interact with a biopsy system and an ultrasound system. The combined user interface (400) may be displayed via a single screen or single display, such as the display (206) of FIG. 4. As has been described, it may be advantageous to provide an interface such as the combined user interface (400) via a control module having a single screen or single display, in order to minimize confusion or distraction related to interacting with different systems or features across multiple screens, displays, or interfaces. An ultrasound view (406) shows output from the ultrasound system as a set of live ultrasound images, which may depict portions of a patient's body that are being imaged, portions of a biopsy device present within the patient's body, and tissues that are being targeted for biopsy.

An interface pane (402) may include one or more buttons, controls, menus, or other interface elements that a user may interact with in order to modify or change the configuration of the combined user interface (400) itself (e.g., such as inputs intended for the interface controller (block 324)). This may include configuring the size and position of various elements of the combined user interface (400), such as changing the size or position of the ultrasound view (406).

An ultrasound pane (404) may show output from the ultrasound system such as device status and configuration information, may show one or more buttons, controls, menus, or other interface elements that a user may interact with in order to modify or change the operation of the ultrasound system, or may show both.

A biopsy pane (408) may show output from the biopsy system such as device status and configuration information, may show one or more buttons, controls, menus, or other interface elements that a user may interact with in order to modify or change the operation of the biopsy system, or may show both. As an example, an aperture size control (410) shows a current aperture size for the biopsy device (28), and may be interacted with by a user to change the aperture size. A vacuum power control (412) shows a current vacuum power for the biopsy device (28), and may be interacted with to enable, disable, or change the power of the vacuum. A tissue sample holder control (414) shows a current rotational position and chamber status of the tissue sample holder, and may be interacted with to rotate the tissue sample holder and align a chamber for receiving a biopsy sample. A set of system controls (416) may be interacted with by a user to change other characteristics of the biopsy system, which may include display characteristics, volume and alert characteristics, and operational characteristics.

The combined user interface (400) advantageously combines output and input features from a biopsy system and an ultrasound system into a single, integrated interface where a physician may view the ultrasound view (406), while also having access to information and controls available from the ultrasound pane (404) and the biopsy pane (408) without having to search for a different display, interface, or user input device. The combined user interface (400) may include additional or different features, and may be implemented with other variations, as will be apparent to one of ordinary skill in the art in light of this disclosure. As an example, this may include variations on the size and positions of the ultrasound view (406), interface pane (402), ultrasound pane (404), and biopsy pane (408). As another example, one or more components of the combined user interface (400) may be dynamic components, and may normally be hidden and accessible via selectable tabs, may pop out from an edge of the interface when clicked on or hovered near or over, may pop over other content when clicked on or hovered near or over, and may be flexible configurable by an end-user by changing code, configurations, or by using a “what you see is what you get” interface editor.

It should also be understood that variations on the unitary control module (200), beyond those explicitly described, exist and will be apparent to one of ordinary skill in the art in light of this disclosure. As an example, this may include reorganizing the positions of components such that a component shown as being in the upper module (202) may be in the lower module (204), and vice versa. With reference to FIG. 7, this may include positioning the ultrasound controller (230) in the upper module (202), in order to position the ultrasound controller (230) and its components, such as an ultrasound generator, more proximately to the ultrasound connection (214) and an attached ultrasound transducer.

The exemplary unitary control modules disclosed herein provide advantages beyond integrating multiple device inputs and outputs into a single user interface. As an example, with reference to FIGS. 4, 5, and 7, by positioning the vacuum module (232) and the power supply (235) in the lower module (204), a significant portion of the overall weight of the unitary control module (200) can be positioned low on the cart (220), and centered just above the broad base of the wheels (224), which provides a low and stable center of gravity. As a result of a low center of gravity positioned centrally over the spread wheels (224), the cart (220) may be less prone to tipping during use or movement. Even where it is not possible to perfectly centrally position the center of gravity, a center of gravity that is substantially centered, lowered, or both provides advantages.

As another example, positioning the vacuum module (232) in the lower module (204) will maximize the distance between the vacuum module (232) and a patient, who will typically be positioned on a support table or support structure at a height closer to the upper module (202). The vacuum module (232) may create significant noise during use, and so maximizing the distance from the patient will reduce the patient's perception of and discomfort with the vacuum operation.

As another example, by routing the vacuum hoses through the tubeset connection (216) and through a set of vacuum hoses that may be contained within the adjustable channel member (226), or may run along the adjustable channel member (226) and partially or fully be covered or shielded from view, the removal and transport of tissue and bodily fluids from the biopsy device (28) to the vacuum canister (234) may be hidden from the patient's view. Similarly, containment and disposal of the contents within the vacuum canister (234) occurs below the patient support structure, and may be hidden from the patient's view. By routing tissue and bodily fluids to the vacuum canister (234) out of the patient's sight, their perception of and discomfort related to the removal of tissue and bodily fluids may be reduced.

III. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### EXAMPLE 1

A unitary control module comprising: (a) a display and a user input device; (b) an ultrasound system operable to produce ultrasound images from an attached ultrasound transducer; (c) a biopsy system configured to control a biopsy device to retrieve tissue samples from a patient; and (d) a cart adapted to support the display, the user input, the ultrasound system, and the biopsy system; wherein the biopsy system is configured to provide output to the ultrasound controller describing a biopsy system status, and receive inputs from the ultrasound controller to control the biopsy device; and wherein the ultrasound system is configured to provide a combined user interface via the display, the combined user interface including: (i) an ultrasound view configured to show ultrasound images, (ii) a biopsy pane configured to show the biopsy system status, and (iii) an ultrasound pane configured to show an ultrasound system status.

### EXAMPLE 2

The unitary control module example 1, wherein the ultrasound system is further configured to: (i) receive an input from a user via the user input device, the input associated with an interface element of the combined user interface, (ii) determine whether the input is associated with changing a configuration of the ultrasound system or the biopsy system, and (iii) where the input is associated with changing the configuration of the biopsy system, format the input to make the input usable by the biopsy system, and provide the formatted input to the biopsy system.

### EXAMPLE 3

The unitary control module of example 2, wherein the ultrasound system is further configured to, where the input is associated with changing the configuration of the ultrasound system, modify the configuration of the ultrasound system based upon the input.

### EXAMPLE 4

The unitary control module of any one or more of examples 1 through 3, wherein the biopsy pane comprises: (i) an aperture size status and user control, (ii) a vacuum power status and user control, and (iii) a tissue sample holder status and user control.

### EXAMPLE 5

The unitary control module of example 4, wherein the biopsy pane is configured to be selectively hidden or displayed based upon a user input to the combined user interface.

### EXAMPLE 6

The unitary control module of any one or more of examples 1 through 5, the cart further comprising a set of wheels adapted to support and stabilize the cart, wherein the ultrasound system and the biopsy system are positioned on the cart to provide a center of gravity proximate to the set of wheels.

### EXAMPLE 7

The unitary control module of example 6, further comprising a vacuum module of the biopsy system and a power supply positioned proximate to the set of wheels.

### EXAMPLE 8

The unitary control module of any one or more of examples 1 through 7, wherein the user input device is a touchscreen interface of the display.

### EXAMPLE 9

A unitary control module comprising: (a) a display and a user input device; (b) an ultrasound system operable to produce ultrasound images from an attached ultrasound transducer; (c) a biopsy system configured to control a biopsy device to retrieve tissue samples from a patient; (d) an interface controller coupled with the ultrasound system and the biopsy system; and (e) a cart adapted to support the display, the user input, the interface controller, the ultrasound system, and the biopsy system; wherein the interface controller is configured to receive output from the ultrasound system and the biopsy system and provide a combined user interface via the display, the combined user interface including: (i) an ultrasound view configured to show ultrasound images, (ii) a biopsy pane configured to show the biopsy system status, and (iii) an ultrasound pane configured to show an ultrasound system status.

### EXAMPLE 10

The unitary control module of example 9, wherein the interface controller is further configured to: (i) receive an input from a user via the user input device, the input associated with an interface element of the combined user interface, (ii) determine whether the input is associated with changing a configuration of: (A) the ultrasound system, or (B) the biopsy system, (iii) where the input is associated with changing the configuration of the biopsy system, format the input to make the input usable by the biopsy system, and provide the formatted input to the biopsy system, and (iv) where the input is associated with changing the configuration of the ultrasound system, format the input to make the input usable by the ultrasound system, and provide the formatted input to the ultrasound system.

### EXAMPLE 11

The unitary control module of example 10, wherein the interface controller is further configured to, where the input is associated with changing the configuration of the interface controller, modify the configuration of the interface controller based upon the input.

### EXAMPLE 12

A method for providing a unitary control module comprising: (a) positioning a display, a user input device, an ultrasound system, and a biopsy system on a cart; (b) coupling the biopsy system with the ultrasound system; (c) configuring the biopsy system to provide output to the ultrasound system describing a biopsy system status, and receive inputs from the ultrasound controller to control the biopsy system; (d) configuring the ultrasound system to provide a combined user interface via the display, the combined user interface including: (i) an ultrasound view configured to show ultrasound images, (ii) a biopsy pane configured to show the biopsy system status, and (iii) an ultrasound pane configured to show an ultrasound system status.

### EXAMPLE 13

The method of example 12, further comprising configuring the ultrasound system to: (i) receive an input from a user via the user input device, the input associated with an interface element of the combined user interface, (ii) determine whether the input is associated with changing a configuration of the ultrasound system or the biopsy system, and (iii) where the input is associated with changing the configuration of the biopsy system, format the input to make the input usable by the biopsy system, and provide the formatted input to the biopsy system.

### EXAMPLE 14

The method of any one or more of examples 12 through 13, further comprising: (a) positioning a vacuum module of the biopsy on a lower portion of the cart, wherein the lower portion of the cart is below a patient support structure supporting a patient, such that a sound produced by the vacuum module is reduced by the patient support structure and a distance between the patient and the lower module before reaching the patient; and (b) adapting a set of vacuum hoses to transfer fluid from a biopsy device to the vacuum module such that the fluid is not visible to the patient.

### EXAMPLE 15

A method for performing a biopsy comprising: (a) positioning a unitary control module near a patient, the unitary control module including a display, a user input device, an ultrasound system, and a biopsy system positioned on a cart, wherein the ultrasound system is (i) coupled with the biopsy system, (ii) configured to receive a biopsy status from the biopsy system and provide a combined user interface via the display based on the biopsy status and an ultrasound status; (b) coupling an ultrasound transducer with the ultrasound system and positioning the ultrasound transducer on the patient to generate a set of ultrasound images; (c) coupling a biopsy probe with the biopsy system and gathering a tissue sample with the biopsy probe based upon the combined user interface, the combined user interface including: (i) an ultrasound view configured to show the set of ultrasound images, (ii) a biopsy pane configured to show the biopsy status, and (iii) an ultrasound pane configured to show the ultrasound status.

### EXAMPLE 16

A unitary control module comprising: (a) a cart including a channel member, an upper module proximate to the upper portion of the channel member, and a lower module proximate to a lower portion of the channel member; (b) a display and a user input device positioned at the upper module; (c) the upper module, including: (i) a biopsy connection adapted to couple with a biopsy device, (ii) an ultrasound connection adapted to couple with an ultrasound transducer, and (iii) an ultrasound controller including an ultrasound processor, the ultrasound processor configured to control the ultrasound transducer coupled with the ultrasound connection; (d) the lower module including a biopsy controller, the biopsy controller including a biopsy processor, the biopsy processor configured to control the biopsy device coupled with the biopsy connection, wherein the biopsy controller is coupled with the ultrasound controller via a connection within the channel member, and configured to provide output to the ultrasound controller describing a biopsy system status, and receive inputs from the ultrasound controller to control the biopsy device; wherein the ultrasound processor is configured to provide a combined user interface via the display, the combined user interface including: (i) an ultrasound view configured to show ultrasound images, (ii) a biopsy pane configured to show the biopsy system status, and (iii) an ultrasound pane configured to show an ultrasound system status.

### EXAMPLE 17

The unitary control module of example 16, further comprising a vacuum module positioned in the lower module, and operable, by the biopsy controller in response to a user interaction with the combined user interface via the user input device, to provide suction to the biopsy device coupled with the biopsy connection.

### EXAMPLE 18

The unitary control module of example 17, further comprising a vacuum canister coupled with the vacuum module and adapted to fluids suctioned by the biopsy device.

### EXAMPLE 19

The unitary control module of example 18, wherein the biopsy device and the vacuum module are coupled by a vacuum hose within the channel member.

### EXAMPLE 20

The unitary control module of any one or more of examples 16 through 19, wherein the ultrasound processor is further configured to: (i) receive an input from a user via the user input device, the input associated with an interface element of the combined user interface, (ii) determine whether the input is associated with changing a configuration of the ultrasound controller or the biopsy controller, and (iii) where the input is associated with changing the configuration of the biopsy controller, format the input to make the input usable by the biopsy controller, and provide the formatted input to the biopsy controller.

### EXAMPLE 21

The unitary control module of example 20, wherein the ultrasound processor is further configured to, where the input is associated with changing the configuration of the ultrasound controller, modify the configuration of the ultrasound controller based upon the input.

### EXAMPLE 22

The unitary control module of any one or more of examples 16 through 21, the cart further comprising a set of wheels adapted to support and stabilize the cart, wherein the upper module and the lower module are adapted to provide a center of gravity proximate to the set of wheels.

### EXAMPLE 23

The unitary control module of example 22, wherein the lower module comprises a vacuum module and a power supply, and wherein the weight of the lower module is substantially greater than the weight of the upper module.

### EXAMPLE 24

The unitary control module of any one or more of examples 16 through 23, wherein the biopsy pane comprises: (i) an aperture size status and user control, (ii) a vacuum power status and user control, and (iii) a tissue sample holder status and user control.

### EXAMPLE 25

The unitary control module of example 24, wherein the biopsy pane is configured to be selectively hidden or displayed based upon a user input to the combined user interface.

### EXAMPLE 26

The unitary control module of any one or more of examples 16 through 25, wherein the user input device is a touchscreen interface of the display.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

It should be understood that any of the versions of instruments described herein may include various other features in addition to or in lieu of those described above. By way of example only, any of the instruments described herein may also include one or more of the various features disclosed in any of the various references that are incorporated by reference herein. It should also be understood that the teachings herein may be readily applied to any of the instruments described in any of the other references cited herein, such that the teachings herein may be readily combined with the teachings of any of the references cited herein in numerous ways. Other types of instruments into which the teachings herein may be incorporated will be apparent to those of ordinary skill in the art.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

We claim:

1. A unitary control module comprising:
(a) a display and a user input device;
(b) an ultrasound system operable to produce ultrasound images using an ultrasound transducer in communication therewith;
(c) a biopsy system configured to control a biopsy device to retrieve tissue samples from a patient; and
(d) a cart adapted to support the display, the user input, the ultrasound system, and the biopsy system;
the biopsy system being configured to provide output to the ultrasound controller describing a biopsy system status, and receive inputs from the ultrasound controller to control the biopsy device; and
the ultrasound system being configured to provide a combined user interface via the display, the combined user interface including:
(i) an ultrasound view configured to show ultrasound images,
(ii) a biopsy pane configured to show the biopsy system status, and
(iii) an ultrasound pane configured to show an ultrasound system status.

2. The unitary control module of claim 1, the ultrasound system being further configured to:
(i) receive an input from a user via the user input device, the input associated with an interface element of the combined user interface,
(ii) determine whether the input is associated with changing a configuration of the ultrasound system or the biopsy system, and
(iii) where the input is associated with changing the configuration of the biopsy system, format the input to make the input usable by the biopsy system, and provide the formatted input to the biopsy system.

3. The unitary control module of claim 2, the ultrasound system being further configured to, where the input is associated with changing the configuration of the ultrasound system, modify the configuration of the ultrasound system based upon the input.

4. The unitary control module of any one or more of claims 1 through 3, the biopsy pane comprising:
(i) an aperture size status and user control,
(ii) a vacuum power status and user control, and
(iii) a tissue sample holder status and user control.

5. The unitary control module of claim 4, the biopsy pane being configured to be selectively hidden or displayed based upon a user input to the combined user interface.

6. The unitary control module of any one or more of claims 1 through 5, the cart further comprising a set of wheels adapted to support and stabilize the cart, the ultrasound system and the biopsy system being positioned on the cart to provide a center of gravity proximate to the set of wheels.

7. The unitary control module of claim 6, further comprising a vacuum module of the biopsy system and a power supply positioned proximate to the set of wheels.

8. The unitary control module of any one or more of claims 1 through 7, the user input device being a touchscreen interface of the display.

9. A unitary control module comprising:
(a) a cart including a channel member, an upper module proximate to an upper portion of the channel member, and a lower module proximate to a lower portion of the channel member;
(b) a display and a user input device positioned proximate the upper module;
(c) the upper module, including:
(i) a biopsy connection adapted to couple with a biopsy device,
(ii) an ultrasound connection adapted to couple with an ultrasound transducer, and
(iii) an ultrasound controller including an ultrasound processor, the ultrasound processor configured to control the ultrasound transducer coupled with the ultrasound connection;
(d) the lower module including a biopsy controller, the biopsy controller including a biopsy processor, the biopsy processor configured to control the biopsy device coupled with the biopsy connection, the biopsy controller being coupled with the ultrasound controller via a connection within the channel member, and configured to provide output to the ultrasound controller describing a biopsy system status, and receive inputs from the ultrasound controller to control the biopsy device;
the ultrasound processor being configured to provide a combined user interface via the display, the combined user interface including:
(i) an ultrasound view configured to show ultrasound images,
(ii) a biopsy pane configured to show the biopsy system status, and
(iii) an ultrasound pane configured to show an ultrasound system status.

10. The unitary control module of claim 9, further comprising a vacuum module positioned in the lower module, and operable, by the biopsy controller in response to a user interaction with the combined user interface via the user input device, to provide suction to the biopsy device coupled with the biopsy connection.

11. The unitary control module of claim 10, further comprising a vacuum canister coupled with the vacuum module and adapted to receive fluids suctioned by the biopsy device.

12. The unitary control module of claim 11, the biopsy device and the vacuum module being coupled by a vacuum hose within the channel member.

13. The unitary control module of any one or more of claims 9 through 12, the ultrasound processor being further configured to:
(i) receive an input from a user via the user input device, the input associated with an interface element of the combined user interface,
(ii) determine whether the input is associated with changing a configuration of the ultrasound controller or the biopsy controller, and
(iii) where the input is associated with changing the configuration of the biopsy controller, format the input to make the input usable by the biopsy controller, and provide the formatted input to the biopsy controller.

14. The unitary control module of claim 13, the ultrasound processor being further configured to, where the input is associated with changing the configuration of the ultrasound controller, modify the configuration of the ultrasound controller based upon the input.

15. The unitary control module of any one or more of claims 9 through 14, the cart further comprising a set of wheels adapted to support and stabilize the cart, the upper module and the lower module being adapted to provide a center of gravity proximate to the set of wheels.

16. The unitary control module of claim 15, the lower module comprises a vacuum module and a power supply, and the weight of the lower module being substantially greater than the weight of the upper module.

17. The unitary control module of any one or more of claims 9 through 16, the biopsy pane comprising:
(i) an aperture size status and user control,
(ii) a vacuum power status and user control, and
(iii) a tissue sample holder status and user control.

18. A method for providing a unitary control module comprising:
(a) positioning a display, a user input device, an ultrasound system, and a biopsy system on a cart;
(b) coupling the biopsy system with the ultrasound system;
(c) configuring the biopsy system to provide output to the ultrasound system describing a biopsy system status, and receive inputs from the ultrasound controller to control the biopsy system;
(d) configuring the ultrasound system to provide a combined user interface via the display, the combined user interface including:
(i) an ultrasound view configured to show ultrasound images,
(ii) a biopsy pane configured to show the biopsy system status, and
(iii) an ultrasound pane configured to show an ultrasound system status.

19. The method of claim 18, further comprising configuring the ultrasound system to:
receive an input from a user via the user input device, the input associated with an interface element of the combined user interface,
(ii) determine whether the input is associated with changing a configuration of the ultrasound system or the biopsy system, and
(iii) where the input is associated with changing the configuration of the biopsy system, format the input to make the input usable by the biopsy system, and provide the formatted input to the biopsy system.

20. The method of any one or more of claims 18 through 19, further comprising:
(a) positioning a vacuum module of the biopsy on a lower portion of the cart, the lower portion of the cart being below a patient support structure supporting a patient, such that a sound produced by the vacuum module is reduced by the patient support structure and a distance between the patient and the lower module before reaching the patient; and
(b) adapting a set of vacuum hoses to transfer fluid from a biopsy device to the vacuum module such that the fluid is not visible to the patient.

21. A method for performing a biopsy comprising:
(a) positioning a unitary control module near a patient, the unitary control module including a display, a user input device, an ultrasound system, and a biopsy system positioned on a cart, wherein the ultrasound system is
(i) coupled with the biopsy system,
(ii) configured to receive a biopsy status from the biopsy system and provide a combined user interface via the display based on the biopsy status and an ultrasound status;
(b) coupling an ultrasound transducer with the ultrasound system and positioning the ultrasound transducer on the patient to generate a set of ultrasound images;
(c) coupling a biopsy probe with the biopsy system and gathering a tissue sample with the biopsy probe based upon the combined user interface, the combined user interface including:
(i) an ultrasound view configured to show the set of ultrasound images,
(ii) a biopsy pane configured to show the biopsy status, and
(iii) an ultrasound pane configured to show the ultrasound status.

22. A unitary control module comprising:
(a) a display and a user input device;
(b) an ultrasound system operable to produce ultrasound images from an attached ultrasound transducer;
(c) a biopsy system configured to control a biopsy device to retrieve tissue samples from a patient;
(d) an interface controller coupled with the ultrasound system and the biopsy system; and
(e) a cart adapted to support the display, the user input, the interface controller, the ultrasound system, and the biopsy system; wherein the interface controller is configured to receive output from the ultrasound system and the biopsy system and provide a combined user interface via the display, the combined user interface including:
(i) an ultrasound view configured to show ultrasound images,
(ii) a biopsy pane configured to show the biopsy system status, and
(iii) an ultrasound pane configured to show an ultrasound system status.

23. The unitary control module of claim 22, wherein the interface controller is further configured to:
(i) receive an input from a user via the user input device, the input associated with an interface element of the combined user interface,
(ii) determine whether the input is associated with changing a configuration of:
(A) the ultrasound system, or
(B) the biopsy system,
(iii) where the input is associated with changing the configuration of the biopsy system, format the input to make the input usable by the biopsy system, and provide the formatted input to the biopsy system, and
(iv) where the input is associated with changing the configuration of the ultrasound system, format the input to make the input usable by the ultrasound system, and provide the formatted input to the ultrasound system.

24. The unitary control module of claim 23, wherein the interface controller is further configured to, where the input is associated with changing the configuration of the interface controller, modify the configuration of the interface controller based upon the input.

25. The unitary control module of claim 17, wherein the biopsy pane is configured to be selectively hidden or displayed based upon a user input to the combined user interface.

26. The unitary control module of any one or more of claims 9 through 17 and 25, wherein the user input device is a touchscreen interface of the display.
